(19) United States
(12) Patent Application Publication
(10) Pub. No.: US 2005/0232958 A1
(43) Pub. Date: Oct. 20, 2005

(54) Temporary tattoo kit

(76) Inventor: Thomas P. Lee, Chicago, IL (US)

Correspondence Address: DICKINSON WRIGHT PLLC, 1901 L. STREET NW, SUITE 800, WASHINGTON, DC, 20036, US

(21) Appl. No.: 11/104,509

(22) Filed: Apr. 13, 2005

Related U.S. Application Data
(60) Provisional application No. 60/521,378, filed on Apr. 14, 2004.

(51) Int. Cl. A61K 7/00; G06F 17/60

(52) U.S. Cl. 424/401; 705/1

(57) ABSTRACT

A temporary tattoo kit containing a sheet assembly having a temporary tattoo. The assembly may include multiple sheets that include at least one sheet with a celebrity tattoo design and is packaged in a container. A method of assembling a temporary tattoo kit is also included with the steps of obtaining a license from an owner of a celebrity tattoo design. The next step requires manufacturing a sheet assembly that includes at least one celebrity design and packaging the assembly within a container. Further, the invention also includes a method of selling and distributing a temporary tattoo. Locations for sale and distribution may include the use of a self serve kiosk machine that may be located at a variety of different locations such as gas stations, airports, and retail stores.

### BACKGROUND OF INVENTION

1. Field of Invention

The present invention generally relates to a temporary tattoo kit and particularly to a temporary tattoo kit with tattoo sets incorporating celebrity's designs and tattoo sets that may be distributed with several point of purchase options.

2. Description of the Related Art

Tattoos have become a very popular and fashionable body decoration or accessory. Many people like the appearance of a tattoo, but do not want to permanently mark their bodies. Thus, the use of temporary tattoos has also gained popularity as it provides the desired aesthetics while allowing the user to remove and replace the tattoo as his or her needs or preferences change. Temporary tattoos may be available through a tattoo artist or other form of retail and may also be available in kit form.

Additionally, many people may desire a particular tattoo design of a celebrity or public figure such as a musician, actor, sportsman or other famous person. Unfortunately, it may be difficult to find a tattoo artist capable of duplicating specific celebrity tattoo designs. It also may be very costly for a tattoo artist to render his or her own version of the celebrity's tattoo or tattoos. In addition, the creator or owner of a particular tattoo design may have copyright protection against others using a design without permission. Further, as celebrities rise and fall in popularity, so too does the demand and desire to imitate their tattoo designs.

Thus, there is a desire and need in the art to provide a temporary tattoo kit that may provide one or more temporary tattoos that incorporate one or more tattoo celebrity designs. There is a further desire to provide means to purchase of a temporary tattoo kit at remote locations such as through a kiosk machine or other point of purchase options.

### SUMMARY OF INVENTION

Accordingly, the present invention provides a temporary tattoo kit containing one or more temporary tattoos incorporating specific celebrity tattoo designs.

One embodiment of the present invention provides a temporary tattoo kit comprising a sheet assembly having a temporary tattoo. The assembly may include multiple sheets that include at least one sheet with a celebrity tattoo design. The assembly is packaged in a container that has instructions of the suggested use of the assembly contained either on the exterior or interior of the container.

The present invention includes a method of assembling a temporary tattoo kit, comprising the steps of obtaining a license from an owner of a celebrity tattoo design. The next step requires manufacturing a sheet assembly that includes at least one celebrity design and packaging the assembly within a container.

The present invention further includes a method of selling and distributing a temporary tattoo kit by any known means. Locations for sale and distribution may include the use of a self-serve kiosk machine that may be located at a variety of different locations such as gas stations, airports, and retail stores.

Other features of the present invention will become more apparent to persons having ordinary skill the art to which the present inv pertains from the following description and claims taken in conjunction with the accompanying figures.

### BRIEF DESCRIPTION OF DRAWINGS

The foregoing features, as well as other features, will become apparent with reference to the description and figures below, in which like numerals represent like elements, and in which:

FIG. 1 is a front view of a first embodiment of a sheet assembly of the present invention;

FIG. 2 is a side view of the sheet assembly shown in FIG. 1;

FIG. 3 is a front view of a second embodiment of a sheet assembly of the present invention;

FIG. 4 is a side view of the sheet assembly shown in FIG. 3;

FIG. 5 is a front perspective view of a first embodiment temporary tattoo kit of the present invention; and

FIG. 6 is a front perspective view of a second embodiment of a temporary tattoo kit of the present invention.

### DESCRIPTION OF THE INVENTION

The present invention generally relates to a kit to allow a user to imitate the body art of a celebrity or public figure. One embodiment uses a temporary tattoo kit and particularly to a temporary tattoo kit with tattoo sets incorporating celebrity designs and tattoo sets that may be distributed with several point of purchase options.

The present invention may best be practiced using a temporary tattoo kit and include one or more sheets of graphical images in either a decal transfer form, an adhesive sticker form, or other means known in the art and placed for sale in a suitable container to allow easy and attractive retail sales at point of purchase locations such as a kiosk, vending areas at concerts, and the like. One aspect of the present invention is that the images included with the kit include celebrity tattoo designs such as frequently found musicians, actors, athletes and other public persons.

The present invention also involves the distribution of the temporary tattoo kit. This may be achieved through a variety of retail methods including, but not limited to, the use of a kiosk machine at a venue where the celebrity is performing, conventional vending areas at music and sporting events, hobby shops, sports trade shows, sporting goods stores, grocery stores, convenience stores, and the like.

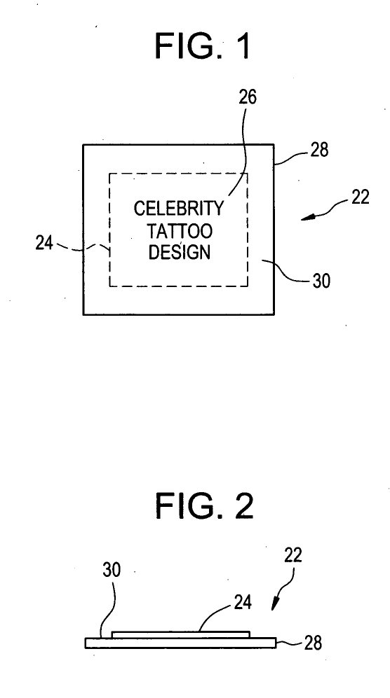

Referring to the figures, a temporary tattoo kit 20 of the present invention may include one or more sheet assembly indicated at 22 on FIG. 1, and indicated at 32 in FIG. 4. Assemblies 22 and 32 may include at least one temporary tattoo 24, 34, which may be in the form of a transferable decal, an adhesive sticker, or other means of conveying body art known in the industry. In the first embodiment (transferable decal form), a sheet assembly 22 may include a base sheet 28 having at least one temporary tattoo 24 printed or otherwise applied to a top surface 30 with, by way of example only, a water soluble ink as shown in FIGS. 1 and 2. Temporary tattoo 24 includes an image 26 that incorporates a design of a tattoo seen on a celebrity. In this embodiment, the water-soluble ink allows for the transfer of image 26 to a person's skin, or other object, when a backside of base sheet 28 is moistened. Such water-soluble temporary tattoos and well known in the art, though other means to transfer graphic images are known and may be used to practice the invention.

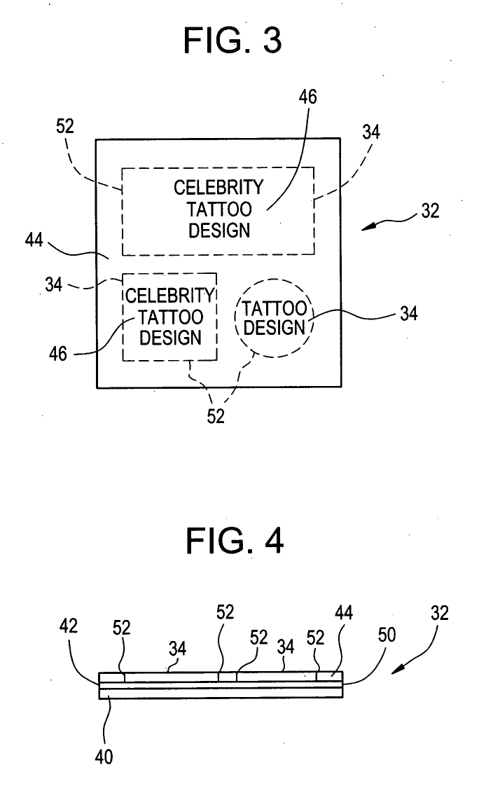

In a second embodiment (adhesive sticker form), a sheet assembly 32 includes a sheet of release paper 40 having an upper surface 42 as shown in FIGS. 3 and 4. Upper surface 42 may include a glossy coating that allows for easy removal of an upper sheet 44. Upper sheet 44 may be adhesively and releasably attached to upper surface 42 with an adhesive layer 50 affixed to a bottom surface of upper sheet 44 as shown in FIG. 4. Upper sheet 44 will contain one or more temporary tattoos 34 having an image 46 corresponding to one or more celebrity tattoos. As with the transferable decal embodiment described above, image 46 incorporates a selected tattoo design seen on a celebrity. The adhesive attachment of upper sheet 44 to release paper 40 allows for upper sheet 44 to be easily removed from upper surface 42 with adhesive layer 50 remaining affixed on a bottom surface of upper sheet 44. The construction of this type of temporary tattoo or sticker is well known in the art. Upper sheet 44 may also include perforated outlines or cutouts 52 to allow a user to remove individual temporary tattoos 34 from upper sheet 44. The adhesive layer remaining on a backside of temporary tattoo 34 allows the user to adhesively affix temporary tattoo 34 to a person's skin or to any other desired object in a typical sticker like manner.

Although temporary, tattoos in the form of transferable decals and adhesive stickers are well known in the art, the present invention provides images 26 and 46 that incorporate known celebrity tattoo designs. It is to be noted that prior to incorporating a particular celebrity tattoo design in kit 20, the manufacturer or marketer of temporary tattoo kit 20 may need to obtain permission or a license from the owner of the particular tattoo design.

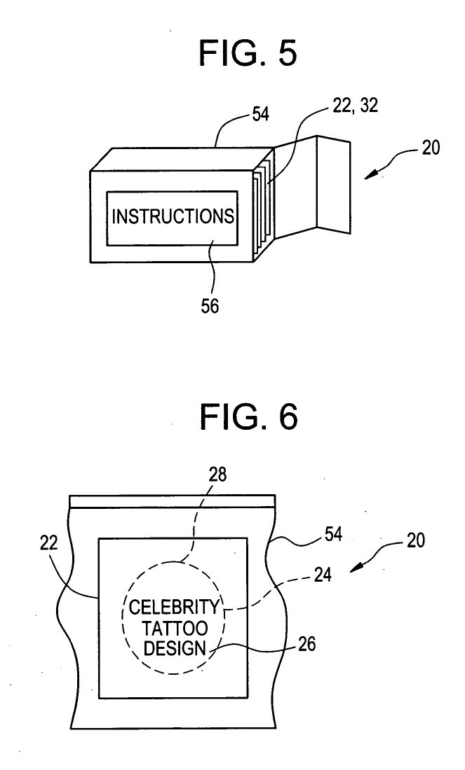

Temporary tattoo kit 20 may include a container 54 (a shown in FIG. 5) that may include a standard box that may be constructed out of any of a variety of known packaging materials, such as cardboard, plastic and metal. Alternatively, container 54 may be in the form of a bag constructed of plastic or paper materials, all of which are well known in the art as shown in FIG. 6.

The present invention further includes any combination of sheet assembly 22 or 32 assembled and positioned within container 54. For example, kit 20 may include one or more sheet assembly 22 having one or more temporary tattoos 24 in the transferable decal form. Alternatively, kit 20 includes one or more of sheet assembly 32 including one or more temporary tattoos 34 in the adhesive sticker form, or a combination of one or more sheet assembly 22 and one or more sheet assembly 32. Kit 20 may include one or more temporary tattoos 24, 34 having images 26, 46 bearing celebrity tattoo designs and may optionally include other temporary tattoos 24, 34 that include non-celebrity tattoo design Further instructions that suggest the use of said assembly may be included. These may be either printed on the box or container or a separate sheet placed “inside the box or container. In addition, kit 20 may include at least one photograph of the celebrity or celebrities displaying the tattoos that are contained in the kit 20.

The kit 20 of the present invention may be sold through any known retail means such as through a self-serve kiosk machine that may be located at a variety of different locations. Kiosk machines are well known in the art and used for sale of a variety of different products. A kiosk machine may be placed in several popular points of purchase locations eliminating the need for the cost of sales associates. For example, a tattoo kit kiosk may be located at gas stations, airports, train and bus stations, retail stores and malls, or any other available public venue allowing customers to purchase desired tattoo kits on a self-serve basis. Alternatively, the kits of the present invention may also be sold by any traditional retail means and the point of purchase may be, by way of example only, concerts, sporting events, or any other venue where a public figure performs.

While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

1. A tattoo kit, comprising:
at least one sheet assembly having a tattoo; said tattoo having a celebrity design;
instructions that suggest the use of said assembly; and a container.

2. The kit of claim 1, wherein said assembly includes a plurality sheets.

3. The kit of claim 1, wherein said kit includes a celebrity photograph.

4. The kit of claim 1, wherein said instructions are contained on the exterior of said container.

5. The kit of claim 1, wherein said instructions are contained in the interior of said container.

6. A method of assembling a temporary tattoo kit, comprising the steps of:
obtaining a license from an owner of at least one celebrity tattoo design;
manufacturing a sheet assembly including at least one said design; and
assembling said assembly within a container.

7. A method of distributing and selling a tattoo kit, comprising the steps of:
obtaining permission to reproduce a specific tattoo design from a celebrity and tattoo artist;
assembling a celebrity tattoo kit, said kit comprising a least one sheet assembly having at least one celebrity tattoo, a container, and instructions;
distributing said kit to a point of purchase; and
selling said kit.

8. The method of claim 7, wherein the step of selling said kit uses a kiosk.

9. The method of claim 7, wherein the point of purchase is selected from the list of gas stations, airports, concerts, sporting events and retail stores.
